(19) United States
(12) Patent Application Publication
(10) Pub. No.: US 2007/0244546 A1
(43) Pub. Date: Oct. 18, 2007

(54) Stent Foundation for Placement of a Stented Valve

(75) Inventor: Richard William Alan Francis, White Bear Lake, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., SANTA ROSA, CA (US)

(21) Appl. No.: 11/379,105

(22) Filed: Apr. 18, 2006

(51) Int. Cl. A61F 2/24; A61F 2/90

(52) U.S. Cl. 623/1.26; 623/2.11; 623/2.18

(57) ABSTRACT

A valve replacement system that can be used for treating abnormalities of the right ventricular outflow tract in a nonsymmetrical region of a vessel or conduit that includes a prosthetic valve device and a foundation structure. The foundation structure contacts a portion of the inner wall of a vessel or conduit, and undergoes a shape change resulting in a corresponding change in the wall of the vessel or conduit. As a result, the lumen of the conduit is made symmetrical, and is complementary to the exterior surface of the stented valve, and thereby, improves the functioning of the valve. Another embodiment of the invention includes a method for replacing a pulmonary valve that includes forming a symmetrical region in a lumen of a conduit and placing a stented valve in the symmetrical region.

### TECHNICAL FIELD

This invention relates generally to medical devices for treating cardiac valve abnormalities, and particularly to a pulmonary valve replacement system and method of employing the same.

### BACKGROUND OF THE INVENTION

Heart valves, such as the mitral, tricuspid, aortic and pulmonary valves, are sometimes damaged by disease or by aging, resulting in problems with the proper functioning of the valve. Heart valve problems generally take one of two forms: stenosis, in which a valve does not open completely or the opening is too small, resulting in restricted blood flow; or insufficiency, in which blood leaks backward across a valve when it should be closed.

The pulmonary valve regulates blood flow between the right ventricle and the pulmonary artery, controlling blood flow between the heart and the lungs. Pulmonary valve stenosis is frequently due to a narrowing of the pulmonary valve or the pulmonary artery distal to the valve. This narrowing causes the right side of the heart to exert more pressure to provide sufficient flow to the lungs. Over time, the right ventricle enlarges, which leads to congestive heart failure (CHF). In severe cases, the CHF results in clinical symptoms including shortness of breath, fatigue, chest pain, fainting, heart murmur, and in babies, poor weight gain. Pulmonary valve stenosis most commonly results from a congenital defect, and is present at birth, but is also associated with rheumatic fever, endocarditis, and other conditions that cause damage to or scarring of the pulmonary valve. Valve replacement may be required in severe cases to restore cardiac function.

Previously, valve repair or replacement required open-heart surgery with its attendant risks, expense, and extended recovery time. Open-heart surgery also requires cardiopulmonary bypass with risk of thrombosis, stroke, and infarction. More recently, flexible valve prostheses and various delivery devices have been developed so that replacement valves can be implanted transvenously using minimally invasive techniques. As a consequence, replacement of the pulmonary valve has become a treatment option for pulmonary valve stenosis.

The most severe consequences of pulmonary valve stenosis occur in infants and young children when the condition results from a congenital defect. Frequently, the pulmonary valve must be replaced with a prosthetic valve when the child is young, usually less than five years of age. However, as the child grows, the valve can become too small to accommodate the blood flow to the lungs that is needed to meet the increasing energy demands of the growing child, and it may then need to be replaced with a larger valve. Alternatively, in a patient of any age, the implanted valve may fail to function properly due to calcium buildup and have to be replaced. In either case, repeated surgical or transvenous procedures are required.

To address the need for pulmonary valve replacement, various implantable pulmonary valve prostheses, delivery devices and surgical techniques have been developed and are presently in use. One such prosthesis is a bioprosthetic, valved conduit comprising a glutaraldehyde treated bovine jugular vein containing a natural, trileaflet venous valve, and sinus. A similar device is composed of a porcine aortic valve sutured into the center of a woven fabric conduit. A common conduit used in valve replacement procedures is a homograft, which is a vessel harvested from a cadaver. Valve replacement using either of these devices requires thoracotomy and cardiopulmonary bypass.

When the valve in the prostheses must be replaced, for the reasons described above or other reasons, an additional surgery is required. Because many patients undergo their first procedure at a very young age, they often undergo numerous procedures by the time they reach adulthood. These surgical replacement procedures are physically and emotionally taxing, and a number of patients choose to forgo further procedures after they are old enough to make their own medical decisions.

Recently, implantable stented valves have been developed that can be delivered transvenously using a catheter-based delivery system. These stented valves comprise a collapsible valve attached to the interior of a tubular frame or stent. The valve can be any of the valve prostheses described above, or it can be any other suitable valve. In the case of valves in harvested vessels, the vessel can be of sufficient length to extend beyond both sides of the valve such that it extends to both ends of the valve support stent.

The stented valves can also comprise a tubular portion or “stent graft” that can be attached to the interior or exterior of the stent to provide a generally tubular internal passage for the flow of blood when the leaflets are open. The graft can be separate from the valve and it can be made from any suitable biocompatible material including, but not limited to, fabric, a homograft, porcine vessels, bovine vessels, and equine vessels.

The stent portion of the device can be reduced in diameter, mounted on a catheter, and advanced through the circulatory system of the patient. The stent portion can be either self-expanding or balloon expandable. In either case, the stented valve can be positioned at the delivery site, where the stent portion is expanded against the wall of a previously implanted prostheses or a native vessel to hold the valve firmly in place.

One embodiment of a stented valve is disclosed in U.S. Pat. No. 5,957,949 titled “Percutaneous Placement Valve Stent” to Leonhardt, et al, the contents of which are incorporated herein by reference.

Over time, implanted prosthetic conduits and valves are frequently subject to calcification, causing the affected conduit or valve to lose flexibility, become misshapen, and fail to function effectively. Furthermore, because they are long term implants, synthetic conduits sometimes undergo longitudinal stretching or fibrotic ingrowth of the tissue surrounding the conduit. In either case, the conduit can become so distorted that blood flow is impeded or the valve is misaligned and fails to function optimally because it is no longer perpendicular to the flow of blood through the conduit.

An additional drawback of using a stented valve is that the stents are often difficult to properly position within a conduit resulting in a misplaced valve. Additionally, stented valves may migrate along the conduit after implantation due to forces applied by the blood flow through the vessel.

It would be desirable, therefore, to provide an implantable pulmonary valve that can readily be replaced, and that would overcome the limitations and disadvantages inherent in the devices described above.

### SUMMARY OF THE INVENTION

It is an object of the present invention to provide a vascular valve replacement system for replacing valves in previously implanted valved conduits, where at least a portion of the conduit has become non-symmetrical after the conduit was implanted. The valve replacement system of the current invention has at least a delivery catheter and a replacement valve device disposed on the delivery catheter. The replacement valve device includes a prosthetic valve connected to a valve support region of an expandable support structure. The valve support region includes a plurality of protective struts disposed between a first stent region and a second stent region.

The system and the prosthetic valve will be described herein as being used for replacing a pulmonary valve. The pulmonary valve is also known to those having skill in the art as the “pulmonic valve” and as used herein, those terms shall be considered to mean the same thing.

Thus, one aspect of the present invention provides a system for treating abnormalities of the right ventricular outflow tract comprising a conduit having a nonsymmetrical lumen, a delivery catheter, a foundation structure, and a prosthetic valve device. The prosthetic valve device comprises a valve connected to a stent. When the foundation structure and the valve device are deployed from the catheter and positioned within the lumen of the conduit, the support structure provides a symmetrical region within the lumen of the conduit that is complementary to the exterior surface of the prosthetic valve device and thereby improves the functioning of the valve.

Another aspect of the invention provides a pulmonary valve replacement system for use in a conduit with a nonsymmetrical lumen. The system includes a foundation structure and a prosthetic valve device. When the foundation structure is positioned within a nonsymmetrical region of the conduit, the foundation structure expands causing a region of the lumen of the conduit to undergo a corresponding shape change. As a result, the affected region of the lumen of the conduit becomes round and symmetrical, and is complementary to the exterior surface of the prosthetic valve device.

Another aspect of the invention provides a method for replacing a pulmonary valve. The method comprises using a catheter to deliver a foundation structure and a pulmonary valve device to a treatment site within the lumen of a conduit. The method further comprises deploying the foundation structure from the catheter within a nonsymmetrical region of the lumen of the conduit. The foundation structure expands and causes a symmetrical region to be formed within the lumen of the conduit. The method further comprises deploying the valve device from the catheter, positioning the valve device within the symmetrical region of the lumen of the conduit.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a schematic interior view of a human heart showing the functioning of the four heart valves;

FIG. 2A is a schematic view showing the placement of a pulmonary conduit, as is known in the prior art;

FIG. 2B is a schematic view showing attachment of a pulmonary conduit to the pulmonary artery, as is known in the prior art;

FIG. 2C is a schematic view showing attachment of a pulmonary conduit to the heart, as is known in the prior art;

FIG. 3 is a schematic view of a delivery catheter with foundation structure and a stented valve device positioned in a nonsymmetrical region of a conduit, in accordance with the present invention;

FIG. 4 is a schematic view of a foundation structure forming a symmetrical fluid passageway complementary to the exterior surface of the stented valve at the treatment site within the lumen of a conduit, in accordance with the present invention;

FIG. 5A is a schematic diagram of a foundation structure having a bracket for holding the valve device in a fixed position, in accordance with the present invention;

FIG. 6A is a schematic diagram of a foundation structure having a holding member on the inner surface of the foundation structure;

FIG. 6B is a cross sectional end view of the foundation structure having a holding member shown in FIG. 6A;

FIG. 7 is a schematic view of a valve support structure in a portion of a conduit that has been restored to a symmetric shape by implanting a tubular scaffold, in accordance with the present invention; and

FIG. 8 is a flow diagram of a method of treating right ventricular outflow tract abnormalities by replacing a pulmonary valve in the lumen of a nonsymmetrical conduit, in accordance with the present invention.

### DETAILED DESCRIPTION

The invention will now be described by reference to the drawings wherein like numbers refer to like structures.

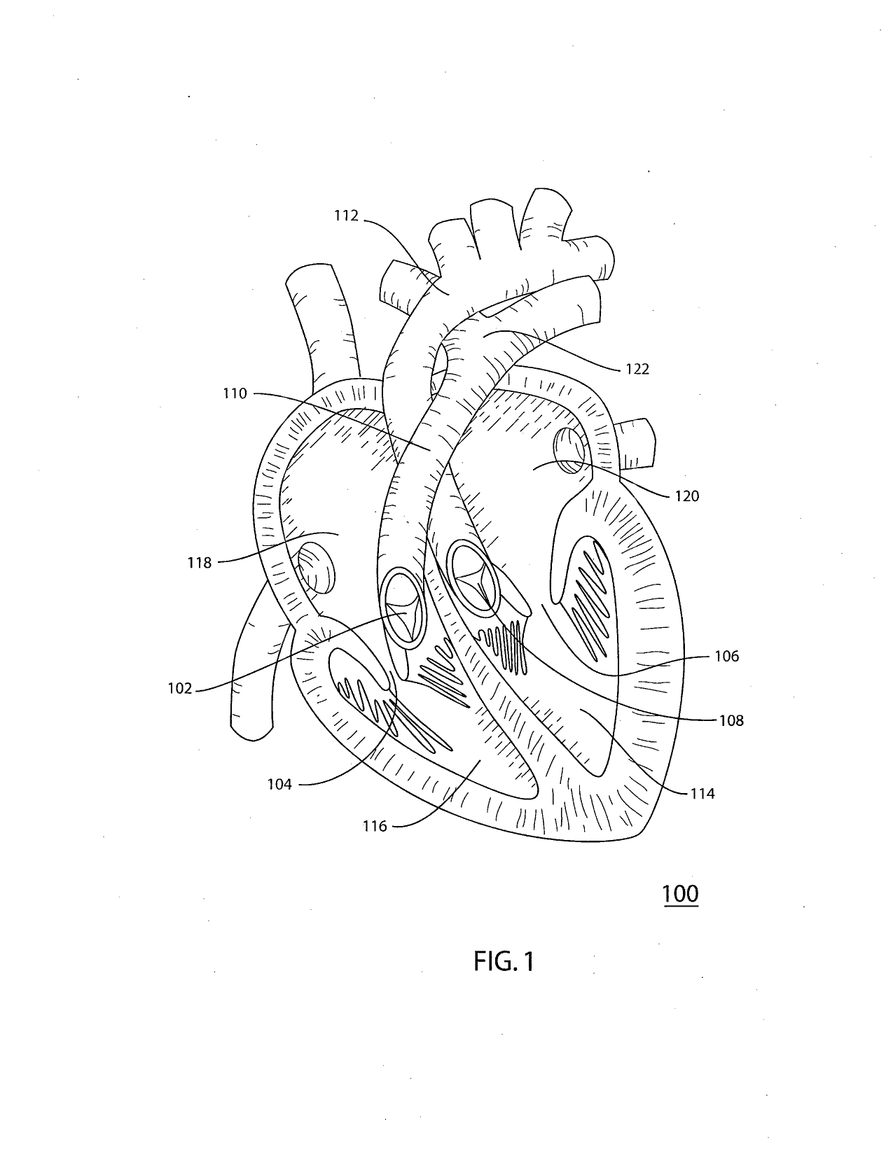

Referring to the drawings, FIG. 1 is a schematic representation of the interior of human heart 100. Human heart 100 includes four valves that work in synchrony to control the flow of blood through the heart. Tricuspid valve 104, situated between right atrium 118 and right ventricle 116, and mitral valve 106, between left atrium 120 and left ventricle 114 facilitate filling of ventricles 116 and 114 on the right and left sides, respectively, of heart 100. Aortic valve 108 is situated at the junction between aorta 112 and left ventricle 114 and facilitates blood flow from heart 100, through aorta 112 to the peripheral circulation.

Pulmonary valve 102 is situated at the junction of right ventricle 116 and pulmonary artery 110 and facilitates blood flow from heart 100 through the pulmonary artery 110 to the lungs for oxygenation. The four valves work by opening and closing in harmony with each other. During diastole, tricuspid valve 104 and mitral valve 106 open and allow blood flow into ventricles 114 and 116, and the pulmonic valve and aortic valve are closed. During systole, shown in FIG. 1, aortic valve 108 and pulmonary valve 102 open and allow blood flow from left ventricle 114, and right ventricle 116 into aorta 112 and pulmonary 110, respectively.

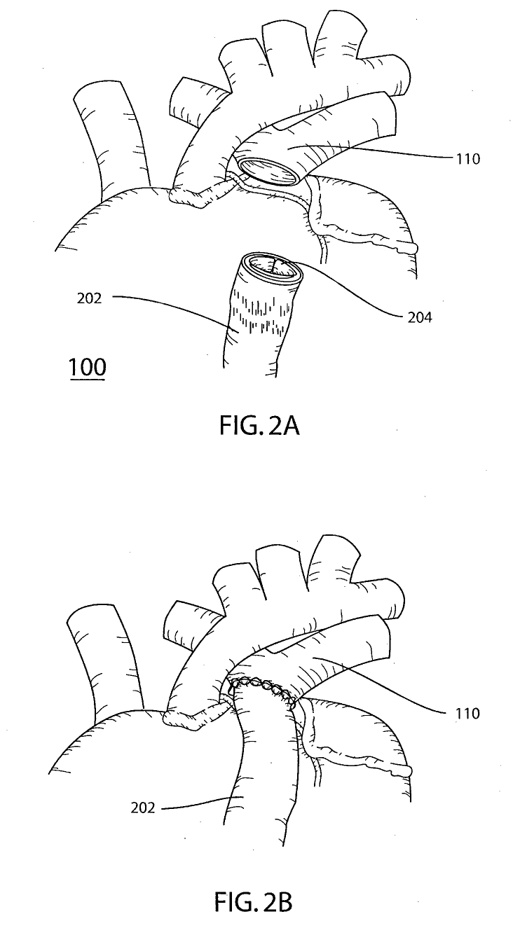

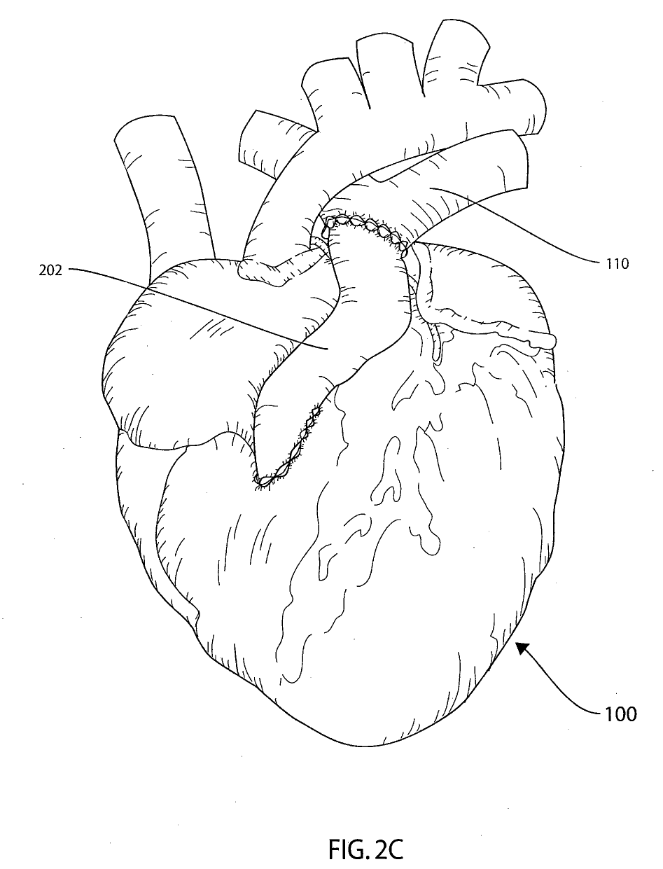

The right ventricular outflow tract is the segment of pulmonary artery 110 that includes pulmonary valve 102 and extends to branch point 122, where pulmonary artery 110 forms left and right branches that carry blood to the left and right lungs respectively. A defective pulmonary valve or other abnormalities of the pulmonary artery that impede blood flow from the heart to the lungs sometimes require surgical repair or replacement of the right ventricular outflow tract with prosthetic conduit 202, as shown in FIG. 2A-C.

Such conduits comprise tubular structures of biocompatible materials, with a hemocompatible interior surface. Examples of appropriate biocompatible materials include polytetrafluoroethylene (PTFE), woven polyester fibers such as Dacron® fibers (E.I. Du Pont De Nemours & Co., Inc.), and bovine vein cross-linked with glutaraldehyde. One common conduit is a homograft, which is a vessel harvested from a cadaver and treated for implantation into a recipient's body. These conduits may contain a valve at a fixed position within the interior lumen of the conduit that functions as a replacement pulmonary valve. One such conduit 202 comprises a bovine jugular vein with a trileaflet venous valve preserved in buffered glutaraldehyde. Other valves are made of synthetic materials and are attached to the wall of the lumen of the conduit. The conduits may also include materials having a high X-ray attenuation coefficient (radiopaque materials) that are woven into or otherwise attached to the conduit, so that it can be easily located and identified.

As shown in FIGS. 2A and 2B, conduit 202, which houses valve 204 within its inner lumen, is installed within a patient by sewing the distal end of conduit 202 to pulmonary artery 110, and, as shown in FIG. 2C, attaching the proximal end of conduit 202 to heart 100 so that the lumen of conduit 202 connects to right ventricle 116.

Over time, implanted prosthetic conduits and valves are frequently subject to calcification, causing the affected conduit or valve to lose flexibility, become misshapen, and lose the ability to function effectively. Additional problems are encountered when prosthetic valves are implanted in young children. As the child grows, the valve will ultimately be too small to handle the increased volume of blood flowing from the heart to the lungs. In either case, the valve needs to be replaced.

The current invention discloses devices and methods for percutaneous catheter based placement of stented valves for regulating blood flow through a pulmonary artery. In a preferred embodiment, the valves are attached to an expandable support structure and they are placed in a valved conduit that is been attached to the pulmonary artery, and that is in fluid communication with the right ventricle of a heart. The support structure can be expanded such that any pre-existing valve in the conduit is not disturbed, or it can be expanded such that any pre-existing valve is pinned between the support structure and the interior wall of the conduit.

The delivery catheter carrying the stented valve is passed through the venous system and into a patient's right ventricle. This may be accomplished by inserting the delivery catheter into either the jugular vein or the subclavian vein and passing it through superior vena cava into right atrium. The catheter is then passed through the tricuspid valve, into right ventricle, and out of the ventricle into the conduit. Alternatively, the catheter may be inserted into the femoral vein and passed through the common iliac vein and the inferior vena cava into the right atrium, then through the tricuspid valve, into the right ventricle and out into the conduit. The catheters used for the procedures described herein may include radiopaque markers as are known in the art, and the procedure may be visualized using fluoroscopy, echocardiography, ultrasound, or other suitable means of visualization.

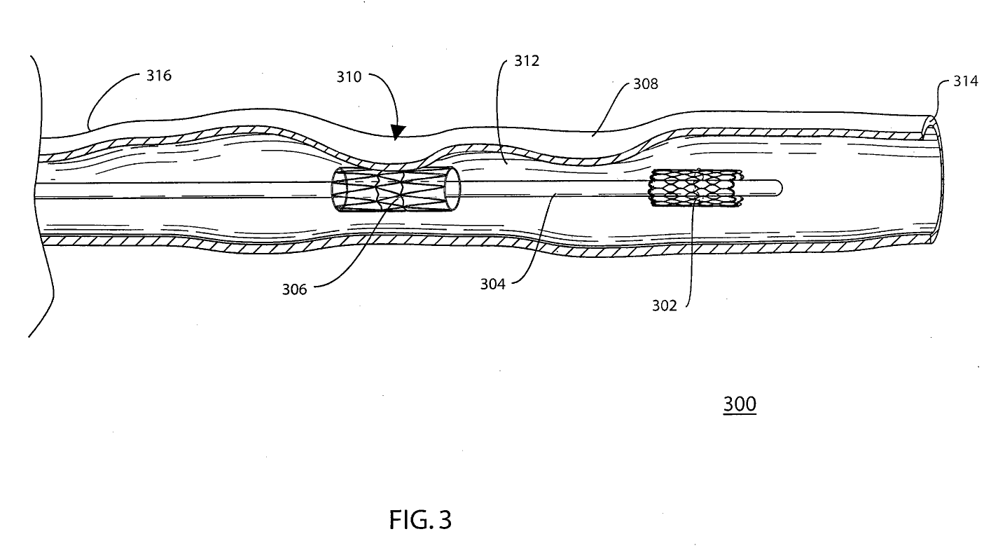

FIG. 3 is a cross-sectional side view of pulmonary valve replacement system 300, having a catheter delivered support structure in accordance with the present invention. Conduit 308 comprises an elongate tubular structure that includes an inner wall that defines lumen 312. Lumen 312 allows fluid communication between right ventricle 116 and pulmonary artery 122. Conduit 308 includes a first end 314 for attaching to ventricle 116 and a second end 316 for attaching to pulmonary artery 122. Stented valve 302 comprises a collapsible valve attached to the interior of a tubular stent.

The stent portion is reduced in diameter and stented valve 302 is mounted on catheter 304. Support structure 306 comprises a flexible material and is also capable of assuming a reduced diameter, being mounted on delivery catheter 304, advanced through the circulatory system of the patient and delivered to treatment site 310, within lumen 312 of conduit 308 as shown in FIG. 3.

In one preferred embodiment, the stented valve 302 and support structure 306 are balloon expandable. In another embodiment, the stented valve and support structure can be self-expanding or a combination of balloon expandable and self-expanding. In the embodiment depicted in FIG. 3, support structure 306 is a tubular scaffold comprising a metallic material or alloy. Examples of suitable metali materials and alloys include, but are not limited to, stainless steel, titanium, platinum, a nickel-titanium alloy, nitinol, iridium, platinum-iridium alloy, gold, tantalum, niobium, and other medically acceptable metals, alone or in combination. In one embodiment of the invention, the body of tubular scaffold 306 comprises a shape memory material such as nitinol, and is self-expanding.

After a conduit 308 has been implanted, it may become calcified or stretch over time. This stretching or calcification can result in a treatment site 310 that is not round and symmetrical. As a result, it may be difficult or impossible to position stented valve 302 in a fixed position, perpendicular to the direction of blood flow within vascular conduit 308, as required for the optimal functioning of stented valve 302. In one embodiment of the invention, the distal portion of catheter 304 is positioned so that support structure 306 is adjacent to treatment site 310, as shown in FIG. 3. When deployed from catheter 304, tubular scaffold 306 expands in diameter and presses against the interior wall of conduit 308 adjacent treatment site 310.

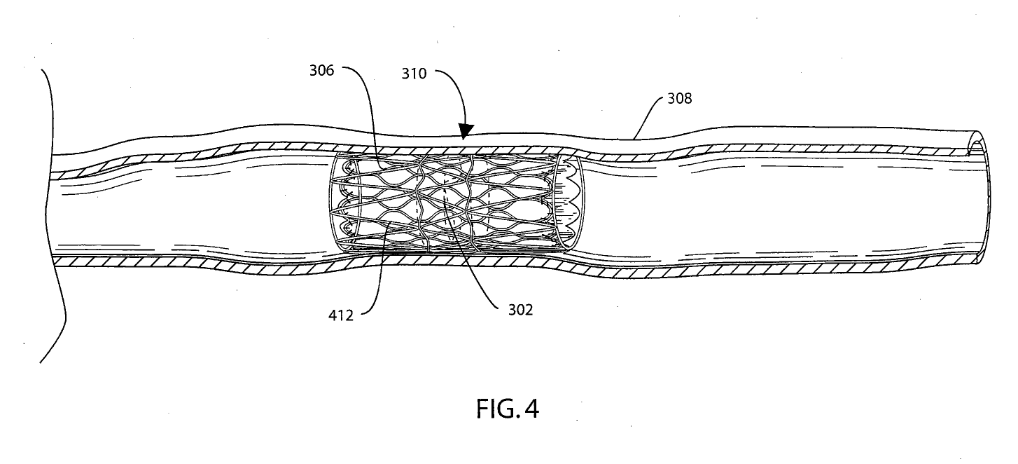

In this embodiment, tubular scaffold 306 has sufficient mechanical strength to reshape the region of the interior lumen of conduit 308 contacted by tubular scaffold 306, as shown in FIG.4. A cylindrical fluid passageway 412, having a constant diameter is formed through the lumen 312 of conduit 308, including treatment site 310. In one embodiment of the invention, the exterior surface of stented valve 302 is cylindrical and is complementary to the cylindrical fluid passageway 412 formed by tubular scaffold 306. Consequently, when stented valve 302 is deployed from catheter 304, within the cylindrical passageway 412 formed by tubular scaffold 306, as shown in FIG.4, the exterior surface of stented valve 302 contacts the inner surface of support structure 306 in close proximity to the wall of the lumen of conduit 308, and is aligned perpendicularly to the flow of blood through conduit 308, and thus improves the functioning of stented valve 302.

In one embodiment of the invention, the exterior surface of the metallic body of support structure 306 is coated with a biostable polymeric material that is nonthrombogenic such as polypropylene, polyethylene, polyurethane, nylon, polytetrafluroethylene (PTFE), and polyester.

To facilitate visualization using fluoroscopy during delivery and accurate placement of support structure 306 within conduit 308, in one embodiment of the invention, at least a portion of support structure 306 comprises a radiopaque material such as, for example, gold, tantalum, and iridium.

In one embodiment, support structure 306 is capable of delivering one or more drugs. In this embodiment, the metallic body of support structure 306 is coated with at least one drug substance such as an anticoagulant drug, antiplatelet drug, anti-inflammatory drug or other drug substance. In one embodiment, the drug substance is mixed with one or more bioabsorbable polymers such polyphosphate ester, polyhydroxybutyrate valerate, and poly (L-lactic acid) to form a uniform coating on the exterior surface of support structure 306 that erodes over a defined period of time and releases the drug substance.

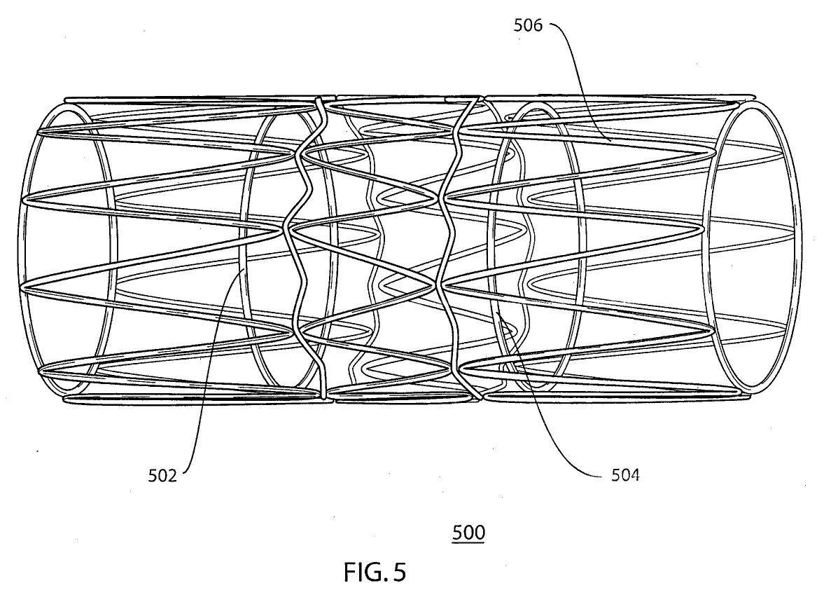

One embodiment of the invention includes a holding means on the interior surface of the support structure. The purpose of the holding means is to prevent migration of stented valve 302 along conduit 308 after implantation due to forces applied by the blood flow through conduit 308. FIG. 5 is a schematic representation of support structure 500 with a bracket. In this embodiment, the tubular body of support structure 506 is substantially the same as support structure 306, but additionally, includes two ring members 502 and 504 located on the inner surface of support structure 506. Ring members 502 and 504 are either molded in the inner surface of support structure 506 or are securely attached to the inner surface of support structure 506. Ring members 502 and 504 are spaced apart so that the distance between ring members 502 and 504 is substantially the same as the length of stented valve 302. When stented valve 302 is delivered between ring members 502 and 504 and expanded against the inner surface of support structure 506, stented valve 302 is held in place and prevented from migrating along the length of the conduit.

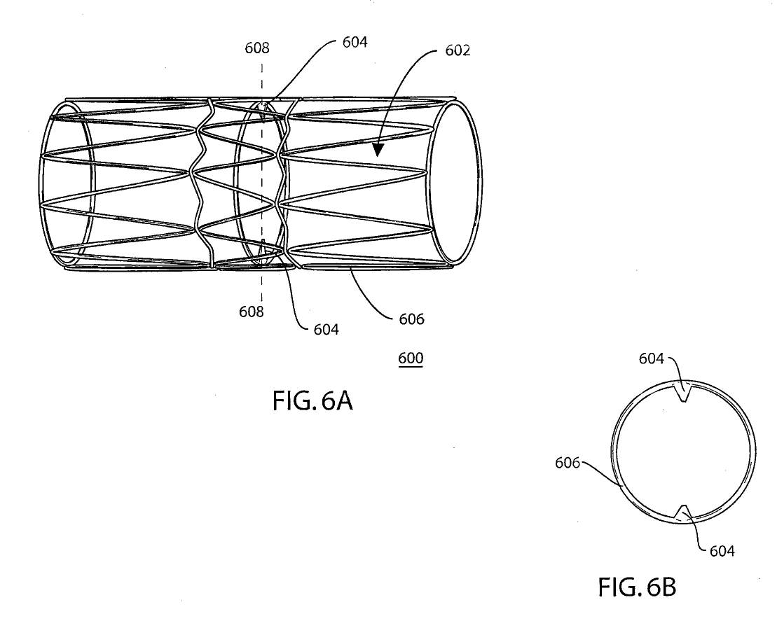

FIGS. 6A and 6B portray another embodiment of the invention. Device 600 includes a holding means that comprises at least one mating portion 604 attached to the interior surface 606 of support structure 602. The embodiment portrayed in FIG. 6A includes two mating portions 604. FIG. 6B provides a cross sectional view of support structure 600 taken at 608-608 in FIG. 6A. In this embodiment, there are two complementary receiving portions in the stent portion of stented valve 302. When stented valve 302 is expanded in the interior lumen of support structure 600, the mating portions 604 pass through the complementary receiving portions of stented valve 302 and maintain stented valve 302 in a fixed position within the interior lumen of support structure 602. In one embodiment of the invention, mating portions 604 are cleats and the complementary receiving portions in stented valve 302 are slots that engage the cleats and maintain the stented valve in a fixed position. In one embodiment, the complementary fit between mating portions 604 and the receiving portions comprises a snap fit. In another embodiment of the invention, stented valve 302 is sutured to the interior wall of support structure 602.

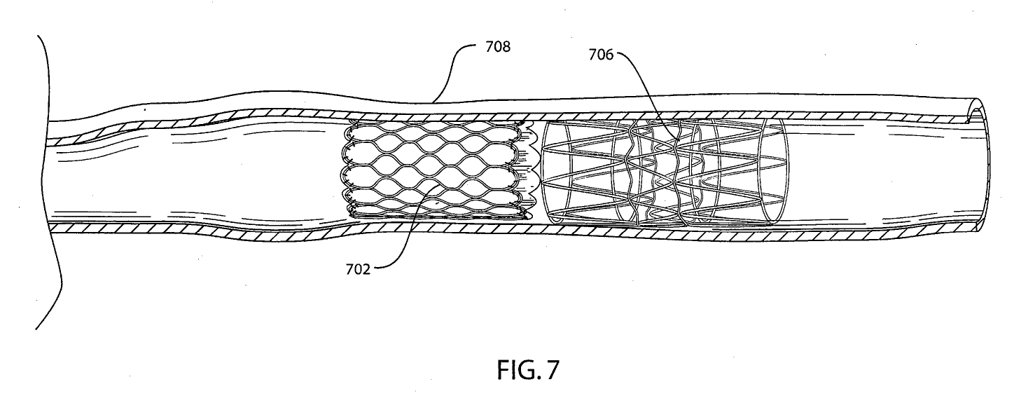

FIG. 7 illustrates that, in some preferred embodiments of the current invention, a stented valve device 702 does not have to be implanted directly into the interior of the foundation structure. Instead, the valve is implanted in any symmetrical portion of the conduit whether that is completely inside of, partially inside of, or completely outside of the tubular scaffold or other foundation structure. In the depicted embodiment, the valve support structure 702 is implanted in an area of the conduit 708 that was restored to a symmetric shape after the tubular scaffold 706 was deployed. For the embodiment depicted, the stented valve is shown deployed on the proximal side (relative to the deploying clinician) of the scaffold. In other embodiments, the valve may be implanted on the distal side of the scaffold, or it may be implanted such that the valve support structure is partially in the scaffold. In another embodiment (not depicted), two or more scaffolds are used to restore the conduit to symmetry and the valve can be implanted in any symmetrical portion of the conduit as described above.

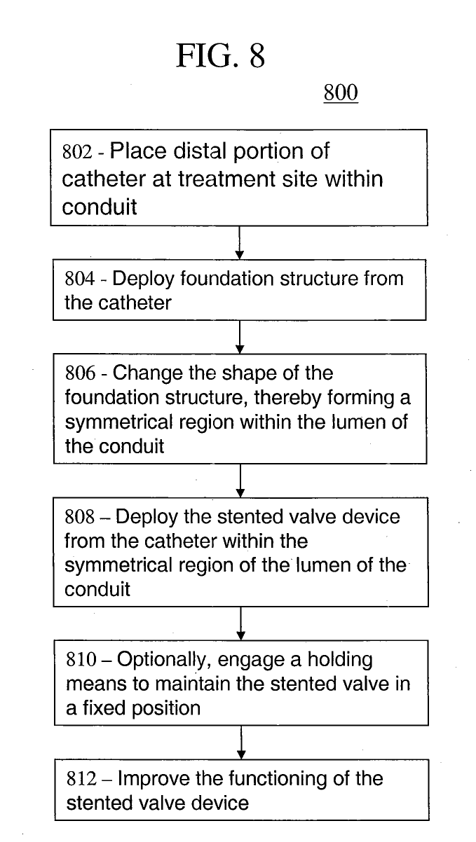

FIG. 8 is a flowchart illustrating method 800 for treating right ventricular outflow tract abnormalities by replacing a pulmonary valve in a nonsymmetrical region of a conduit, in accordance with the present invention. Beginning at Block 802, a foundation structure (such as foundation structure 306 or 506) and a stented valve (such as stented valve 302) are mounted on a catheter such as catheter 304. The distal portion of delivery catheter 304 is then passed through the venous system and into a patient's right ventricle 116. This may be accomplished by inserting delivery catheter 304 into either the jugular vein or the subclavian vein, and passing it through the superior vena cava into right atrium 118. The catheter is then passed through tricuspid valve 104, into right ventricle 116, and out of the ventricle into conduit 308. Alternatively, delivery catheter 304 may be inserted into the femoral vein and passed through the common iliac vein and the inferior vena cava into right atrium 118, then through tricuspid valve 104, into right ventricle 116, and out into conduit 308. The catheters used for the procedures described herein may include radiopaque markers as is known in the art, and the procedure may be visualized using fluoroscopy, echocardiography, ultrasound, or other suitable means of visualization.

Next, a foundation structure is deployed from the catheter at the treatment site within a non-symmetric region of either a prosthetic lumen such as lumen 312 or a deformed blood vessel, as indicated in Block 804. A foundation structure such as foundation structure 306 or 506 may be used. The foundation structure is expanded in diameter so that the exterior surface of the foundation structure presses against the interior wall of conduit 308 and reshapes a region of the inner lumen of conduit 308. In this embodiment, tubular scaffold 306 has sufficient mechanical strength to reshape the region the interior lumen of conduit 308 contacted by the support structure. As a consequence, the inner lumen of conduit 308 forms a symmetrical region of uniform diameter surrounding the support structure, as indicated in Block 806.

Next, a stented valve such as stented valve 302 is deployed from the delivery catheter into symmetrical region within the lumen of conduit 308, as indicated in Block 808. The stented valve is expanded, and if a foundation structure such as foundation structure 506 is used, stented valve 302 is positioned so that a mating portion of a holding means on the foundation structure engages a receiving portion on the exterior surface of the stented valve (Block 810). In one embodiment, stented valve 302 is positioned between first and second ring members and expanded. In either case, stented valve 302 is maintained in a fixed position by the holding means within the symmetrical region of conduit 308, and aligned perpendicular with the flow of blood, which allows the valve to function optimally (Block 812).

While the invention has been described with reference to particular embodiments, it will be understood by one skilled in the art that variations and modifications may be made in form and detail without departing from the spirit and scope of the invention.

What is claimed is:

1. A vascular valve replacement system for use in a conduit having a nonsymmetrical lumen, the system comprising:
a conduit having a lumen at least a portion of which is nonsymmetrical;
a delivery catheter;
a foundation structure; and
a prosthetic valve device including a valve connected to a stent;
the foundation structure and the prosthetic valve device disposed on the catheter;
wherein when the foundation structure and the valve device are deployed from the catheter in the nonsymmetrical portion of the lumen of the conduit, the foundation structure provides a symmetrical region within the lumen of the conduit complementary to the exterior surface of the prosthetic valve device and thereby improves the functioning of the valve device.

2. The system of claim 1 wherein the foundation structure is a tubular scaffold that defines a cylindrical fluid passageway when expanded through a nonsymmetrical portion of the lumen of the conduit.

3. The system of claim 2 wherein the tubular scaffold is either balloon expandable or self-expanding.

4. The system of claim 2 wherein the tubular scaffold comprises a metallic material selected from a group consisting of stainless steel, titanium, platinum, iridium, gold, nickel-titanium alloy, nitinol, platinum-iridium alloy, tantalum, niobium and combinations thereof.

5. The system of claim 4 wherein at least a portion of the tubular scaffold is radiopaque.

6. The system of claim 4 wherein the metallic material is covered with a biostable polymeric material selected from a group consisting of polypropylene, polyethylene, polyurethane, nylon, polytetrafluroethylene (PTFE), polyester, other medically approved polymers, and combinations thereof.

7. The system of claim 4 wherein the tubular scaffold is coated with a drug-eluting polymer.

8. The system of claim 1 further comprising a holding means on the interior surface of the foundation structure that engages a portion of the stented valve and maintains the stented valve in a fixed position.

9. The system of claim 6 wherein the holding means is selected from a group consisting of a bracket, cleats, a snap fit, and sutures.

10. A pulmonary valve replacement system for use in a conduit having a nonsymmetrical lumen, the system comprising:
a conduit including a nonsymmetrical portion;
a foundation structure; and
a prosthetic valve device including a valve connected to a stent wherein, when the foundation structure is positioned within a nonsymmetrical region of the conduit, the foundation structure expands causing a region of the lumen of the conduit to undergo a shape change, thereby providing a symmetrical region within the lumen of the conduit complementary to the exterior surface of the prosthetic valve device.

11. A method of replacing a pulmonary valve, the method comprising:
delivering a foundation structure and a prosthetic valve device to a treatment site within a lumen of a conduit via catheter;
deploying the foundation structure from the catheter;
expanding the foundation structure;
forming a symmetrical region within the lumen of the conduit; and
deploying the prosthetic valve device from the catheter in the interior of the foundation structure within the symmetrical region within the lumen of the conduit.

12. The method of claim 11 wherein the foundation structure is a tubular scaffold having an interior lumen and wherein forming a symmetrical region further comprises forming a cylindrical fluid passageway through a portion of the conduit.

13. The method of claim 12 further comprising:
expanding the tubular scaffold to engage the interior surface of the conduit, thereby causing a region of the conduit to assume a round, symmetrical shape complementary to the exterior surface of the valve device.

14. The method of claim 11 further comprising:
positioning the prosthetic valve device within the interior lumen of the tubular scaffold.

15. The method of claim 14 further comprising:
engaging a holding means on the interior surface of the tubular scaffold with a portion of the prosthetic valve device; and
securing the prosthetic valve device in a fixed position.

16. The method of claim 15 wherein engaging a holding means further comprises engaging a clip or cleat on the interior surface of the tubular scaffold with a complementary receiving portion on the exterior surface of the prosthetic valve device.

17. The method of claim 15 wherein engaging a holding means further comprises forming at least one suture between the tubular scaffold and the stent portion of the stented valve device.

18. The method of claim 15 wherein engaging a holding means further comprises positioning the stented valve device against a bracket on the interior surface of the tubular scaffold.

19. The method of claim 11 further comprising releasing an antithrombic drug from a drug-delivery coating on the exterior surface of the tubular scaffold and preventing thrombosis.

20. The method of claim 11 further comprising improving the functioning of the prosthetic valve by providing a round, symmetrical fluid passageway through a portion of the lumen of the conduit.
